(19) United States
(12) Patent Application Publication
(10) Pub. No.: US 2026/0325794 A1
(43) Pub. Date: Oct. 22, 2026

(54) MEDICAL TUBE MANAGEMENT DEVICE

(71) Applicant: Conrad David Charles, Glen Allen, VA (US)

(72) Inventor: Conrad David Charles, Glen Allen, VA (US)

(21) Appl. No.: 19/467,729

(22) Filed: Feb. 2, 2026

Related U.S. Application Data
(60) Provisional application No. 63/752,676, filed on Feb. 1, 2025.

(51) Int. Cl. A61M 25/02

(52) U.S. Cl. CPC A61M 25/02; A61M 2025/024; A61M 2209/082

(57) ABSTRACT

A medical tube management device is described according to one or more embodiments described herein. In an example, a tube holding apparatus can include a base arm that can be rotatably coupled to a support arm. The support arm can have a front side that faces toward the base arm and a back side that faces opposite the front side. The tube holding apparatus can further include a channel that can be positioned on the back side of the support arm, and the channel can house an adjustable plate. The tube holding apparatus can further include one or more clamps that can be attached to the adjustable plate. The one or more clamps can be configured to hold one or more tubes.

### CROSS-REFERENCE TO RELATED APPLICATION(S)

This present application claims priority to U.S. Provisional Patent Application No. 63/752,676, filed Feb. 1, 2025, which is incorporated herein by reference in its entirety.

### TECHNICAL FIELD

This disclosure relates to medical accessories, specifically to a tube management device for organizing and/or securing medical tubing.

### BACKGROUND

Medical treatments and therapies often use various tubes and hoses to deliver oxygen, air, medication, or other therapeutic gases to patients. Devices such as Continuous Positive Airway Pressure (CPAP) and Bilevel Positive Airway Pressure (BiPAP) machines can be prescribed for individuals suffering from sleep apnea and other respiratory conditions. These machines utilize flexible tubes to deliver pressurized air to a patient's airway via a mask. Similarly, other medical equipment, including oxygen concentrators, nebulizers, and feeding systems, also rely on tubing to administer treatment effectively. Often such medical treatments, therapies, and equipment are utilized in a bedside manner while the patient is positioned in a bed.

### SUMMARY

In one embodiment, a tube holding apparatus can include a base arm that can be rotatably coupled to a support arm. The support arm can have a front side that faces toward the base arm and a back side that faces opposite the front side. The tube holding apparatus can include a channel that can be positioned on the back side of the support arm. The channel can house an adjustable plate. The tube holding apparatus can further include one or more clamps that can be attached to the adjustable plate. The one or more clamps can be configured to hold one or more tubes.

In yet another embodiment, a method of managing medical tubing using a tube holding device can include transitioning the tube holding device from a folded configuration to a deployed configuration by rotating a support arm of the tube holding device relative to a base arm of the tube holding device, inserting the base arm beneath a support surface such that the support arm abuts a side thereof, and securing one or more medical tubes within one or more clamps of the tube holding device.

In yet another embodiment, a foldable bedside holder can include a base arm rotatably coupled to a support arm via a hinge and a channel positioned on a back side of the support arm and defined by sidewalls including one or more aligned sets of holes. The channel can house an adjustable plate with at least one through-hole. The foldable beside holder can include one or more clamps coupled to the adjustable plate. The hinge can enable rotation of the support arm relative to the base arm to transition the holder between a folded configuration and a deployed configuration, and in the deployed configuration, the adjustable plate is slidable along the channel to a selected height and securable via a locking mechanism insertable through a selected set of the aligned holes and the at least one through-hole.

These and other features and advantages of the present disclosure will become apparent from the following description of particular embodiments, when viewed in accordance with the accompanying drawings and appended claims.

Before the embodiments of the present disclosure are explained in detail, it is to be understood that the technology is not limited to the details of operation or to the details of construction and the arrangement of the components set forth in the following description or illustrated in the drawings. The technology described herein can be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the technology to any specific order or number of components. Nor should the use of enumeration be construed as excluding from the scope of the technology any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

FIGS. 1A-1B are example perspective views of a tube holding device.

FIGS. 2A-2B are example bottom perspective views of the tube holding device.

FIG. 3 is an example side view of the tube holding device.

FIG. 4 is an example of a top-down view of the tube holding device.

FIGS. 5A-5B depict example front views of the tube holding device.

FIGS. 6A-6B depict example back views of the tube holding device.

FIGS. 7A-7C illustrate example perspective views of the tube holding device in an extended configuration.

FIGS. 8A-8B depict example perspective views of the tube holding device in a folded configuration.

FIGS. 9A-9B illustrate example operational views of the tube holding device.

FIG. 10 depicts an example perspective view of a clamp of the tube holding device.

FIG. 11 depicts an example back view of the tube holding device.

FIG. 12 depicts an example perspective of the tube holding device.

### DETAILED DESCRIPTION

Embodiments/examples of the present disclosure will now be described in detail with reference to the accompanying figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Some medical treatments and therapies use various tubes and/or hoses for fluids, gases, and/or medications. Often these tubes and hoses are employed with medical equipment positioned alongside furniture (e.g., a bed or chair) where a patient is positioned or located. While these tubes are needed for patient care, unmanaged tubing presents numerous challenges. Loose or excess tubing can become tangled, pulled, or pinched during patient movement, resulting in discomfort, mask displacement, air leaks, or complete therapy interruption. Furthermore, tubing that trails across furniture (e.g., a bed) or flooring poses tripping hazards and increases the risk of accidental disconnection or damage.

Existing approaches for managing medical tubing are often cumbersome, lack adaptability, or fail to provide a secure, convenient, and ergonomic solution suitable for in-home furniture use. Additionally, existing holders are designed for hospital environments and thus need permanent mounting to bed frames, walls, or IV poles, rendering them impractical for residential settings. Moreover, poorly designed supports can place undue stress on the tubing, leading to premature wear, kinking, or airflow restriction.

Embodiments in accordance with the present disclosure generally relate to a tube holding device (e.g., bedside device) for managing medical tubing. Advantageously, various embodiments described herein include a device that is versatile, easy to use, and capable of securing various types of tubes without compromising functionality or patient comfort. In accordance with various embodiments described herein, the tube holding device can be employed in conjunction with various types of furniture, such as bed, where a patient may be located while receiving medical treatment. For example, the tube holding device as described herein can be utilized in a bedside manner with patients engaging medical equipment to treat CPAP and/or BiPAP.

In yet some examples, the tube holding device as described herein can be utilized in medical facilities, hospitals, nursing homes, residences, and/or the like to manage tubing associated with various medical equipment. Additionally, in accordance with various embodiments described herein, the tube holding device can be compact, having the ability to fold, unfold, extend, and/or retract to accommodate numerous configurations.

As used herein the term “tube” can refer to a generally elongated, hollow conduit for transporting fluids, gases, and/or air. A tube can be rigid, semi-rigid, or flexible and can be formed from various materials, including metal, plastic, and/or rubber. The tube can have a defined inner and/or outer diameter and can include structural reinforcements to maintain a corresponding shape and/or flexibility. In some examples, the term “tube” as used herein encompasses flexible conduits commonly referred to as hoses, as well as rigid or semi-rigid conduits, and can be used in medical, industrial, or consumer applications.

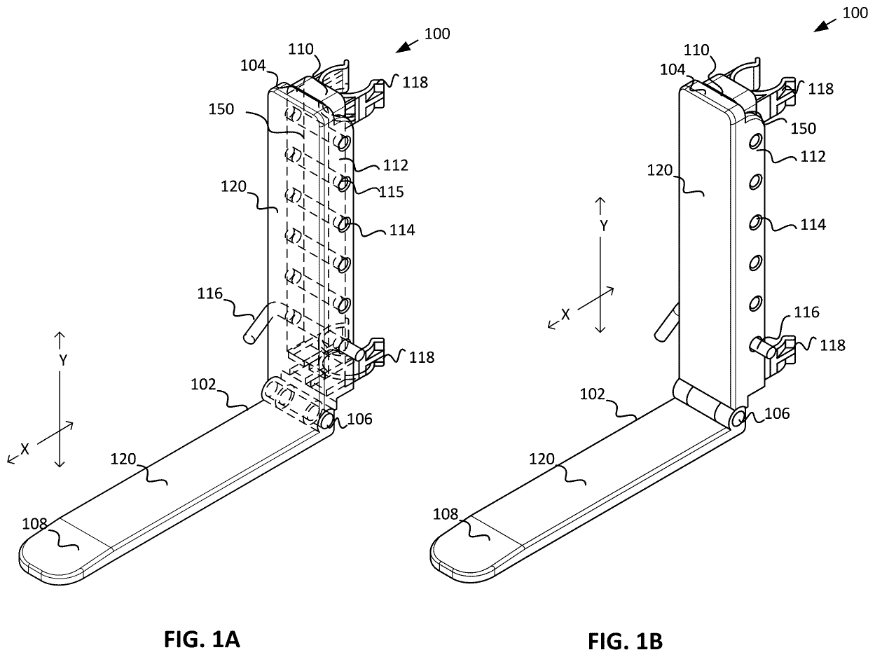

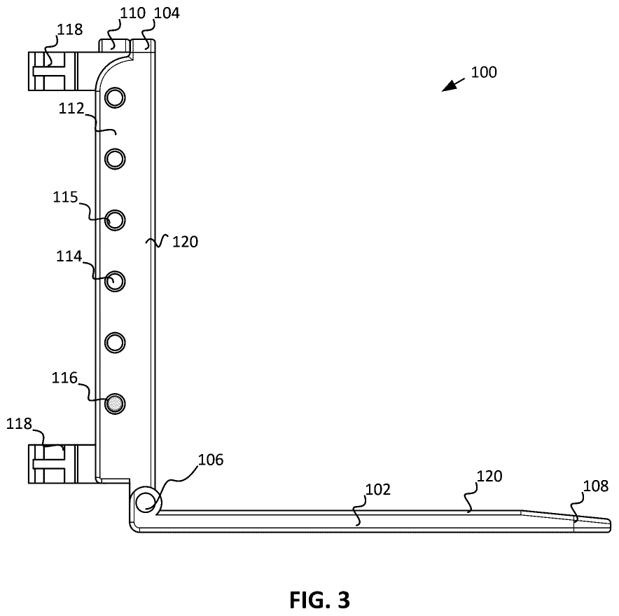

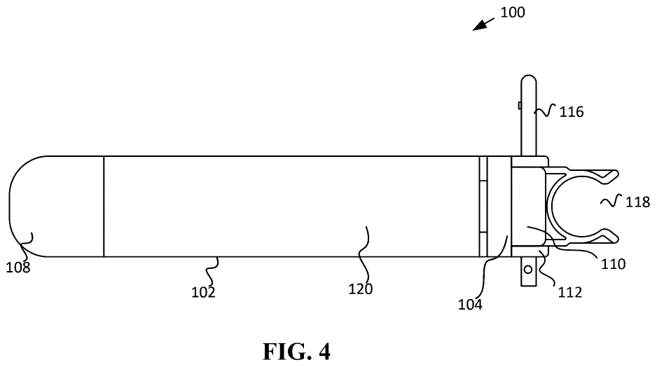

FIGS. 1A-1B depict example top-down perspectives of a tube holding device 100 in accordance with one or more embodiments described herein. FIG. 1A depicts the tube holding device 100 with internal components delineated by dashed lines. FIG. 1B depicts the tube holding device 100 of FIG. 1A with solid surfaces, such that internal components are not visible. As shown in FIGS. 1A-1B, the tube holding device 100 can include a base arm 102 and a support arm 104, which can be coupled together by a pivot (or hinge) 106. Each of the base arm 102 and the support arm 104 can rotate about the pivot 106 while maintaining connection to each other. In various embodiments, the pivot 106 can be a hinge coupled to the base arm 102 and the support arm 104 (e.g., as shown in FIGS. 1A-B). To achieve a folded configuration (e.g., FIG. 4), the base arm 102 and support arm 104 are rotated about the hinge 106 until respective front faces 120 are substantially parallel and adjacent. To achieve a deployed configuration (e.g., FIGS. 1A-1B, 3), the support arm 104 can be rotated about the hinge 106 away from the base arm 102 until the arms form an angle of approximately 90 degrees (or, in other examples, less than or greater than 90 degrees)

FIGS. 1A-1B depicts the tube holding device 100 in an open state or deployed/unfolded configuration, where the base arm 102 and the support arm 104 are perpendicular (e.g., about 90 degrees) to each other. In various embodiments, while in the open state the base arm 102 and the support arm 104 can be positioned at an angle to each other that is less than 90 degrees, or greater than 90 degrees. In some examples, the pivot 106 can include a locking mechanism to enable the base arm 102 and support arm 104 to remain in a fixed position with respect to each other. For example, when engaged, the locking mechanism can mitigate and/or prevent rotation of the base arm 102 and/or the support arm 104 about the pivot 106. The locking mechanism can include a friction-fit hinge, a detent pin (e.g., a spring-loaded detent pin), or a ratchet mechanism integrated into the hinge 106. In yet some examples, the detent pin engages one of a plurality of recesses in the hinge 106 to selectively lock the angle between the base arm 102 and the support arm 104. In some examples, a thumb screw or cam lever can be tightened to increase frictional engagement and maintain the desired configuration.

In some embodiments, the base arm 102 can have a substantially rectangular geometry (e.g., as shown in FIGS. 1A-1B). However, in other examples/embodiments, the base arm 102 can have a different geometry (e.g., a pronged shape, a substantial circular shape, an elliptical shape, a substantially square shape, and/or the like). In some examples, the distal end of the base arm 102 can be tapered (e.g., such as a tapered end 108, as shown in FIGS. 1A-1B). A length of the base arm 102 can be in a range from, for example, but not limited to, 3 inches to 36 inches (although greater or shorter lengths are also envisaged). A width of the base arm 102 can range from, for example, but not limited to, 0.5 inches to 36 inches (although greater or smaller widths are also envisaged). In some examples, the base arm 102, support arm 104, hinge 106, adjustable plate 110, and/or clamps 118 may be formed from metals (e.g., aluminum, steel, stainless steel, titanium, iron, and/or the like), plastics, resins, composite materials, or combinations thereof, and/or the like.

In some examples, the support arm 104 can house an adjustable plate 110. For instance, the adjustable plate 110 can be positioned along a longitudinal axis of the support arm 104 and can be located adjacent to a side of the support arm 104 that faces away from the base arm 102 (e.g., as shown in FIGS. 1A-1B). In some embodiments, the adjustable plate 110 can be positioned within a channel 150 of the support arm 104. For example, longitudinal sidewalls 112 and at least one lateral sidewall of the channel 150 can partially surround the adjustable plate 110.

In some examples, the support arm 104 can include holes 114 aligned that can be aligned with corresponding holes in the adjustable plate 110. For example, the holes 114 can be within the longitudinal sidewalls 112. The corresponding holes 115 of the adjustable plate 110 can traverse in the lateral direction in a configuration that enables alignment with the holes 114 positioned in the longitudinal sidewalls 112. In some examples, the adjustable plate 110 includes at least one transverse through-hole 115 configured to align with a pair of holes (e.g., the holes 114) of the support arm 104 when the plate 110 is positioned at a desired height along a Y-axis, as shown in FIG. 1A. In some examples, this alignment allows a locking pin 116 to be inserted through the sidewalls 112 and the adjustable plate 110, thereby fixing a vertical position of the plate 110 and attached clamps 118. For instance, an example location of the holes 114 is visible in FIG. 1B, and an example trajectory of the holes 114 is delineated by dashed lines in FIG. 1A. While FIGS. 1A and 1B depict six sets of aligned holes 114, fewer or additional sets are also envisaged.

In some examples, the adjustable plate 110 can slide within the channel 150 of the support arm 104 along the longitudinal direction (e.g., along the Y axis shown in FIGS. 1A-1B). For example, in a retracted state, the adjustable plate 110 can be positioned such that a distal end of the adjustable plate 110 is even, or shy of, the distal end of the support arm 104. In an extended state or position, the distal end of the adjustable plate 110 can extend past the distal end of the support arm 104. In yet some examples, positioning of the adjustable plate 110 with respect to the support arm 104 can be secured, at least partially, by the locking mechanism 116 that interacts with the holes 114 of the adjustable plate 110 and the support arm 104.

For example, the locking mechanism 116 can be a pin (e.g., as shown in FIGS. 1A-1B) that extends through the longitudinal sidewalls 112 and the adjustable plate 110 via sets of aligned holes, as described herein. As the adjustable plate 110 is extended from between the longitudinal sidewalls 112, the holes 115 in the adjustable plate 110 can align with a different set of the holes 114 in the longitudinal sidewalls 112, and the locking mechanism 116 can engage this new alignment to secure the extended position. While the locking mechanism 116 is depicted as a pin in FIGS. 1A-1B, in other examples, the locking mechanism can be a barb, a button, a latch, and/or the like.

In some instances, the adjustable plate 110 is depicted in the retracted position in FIGS. 1A-1B, with the locking mechanism 116 engaging a lower-most set of holes 114 and/or 115 along the longitudinal direction (e.g., Y axis); the adjustable plate 110 can traverse along the longitudinal length of the support arm 104 to an extended position, in which the holes 115 of the adjustable plate 110 can align with one or more sets of holes positioned above the lower-most set of holes 114 along the Y-axis. In yet some examples, the tube holding device 100 can be characterized by a compact profile in the retracted state, while enabling a utilization of a larger profile in an extended state when deployed in various tube management scenarios.

In some examples, one or more clamps 118 can be attached to the adjustable plate 110 and/or the support arm 104. The clamps 118 can be utilized to hold tubing in connection with various medical equipment. For example, the one or more clamps 118 can have a semi-rigid construction; being able to flex when accepting a tube and apply a compressive force against the accepted tube to hold the tube in place. Examples of clamps 118 that can be employed include but are not limited to: C-clamps (e.g., as shown in FIGS. 1A-1B), tube clamps, hose clamps, conduit clamps, pipe hangers, a combination thereof, and/or the like. As shown in FIGS. 1A-1B, the one or more clamps 118 can extend from the adjustable plate 110 in a direction away from the front face 120 of the support arm 104. In various embodiments, a plurality of clamps 118 can be attached to the adjustable plate 110. In one or more embodiments, one or more clamps 118 can be attached to the support arm 104 (e.g., positioned below the adjustable plate 110, near the pivot 106), while one or more clamps 118 are attached to the adjustable plate 110.

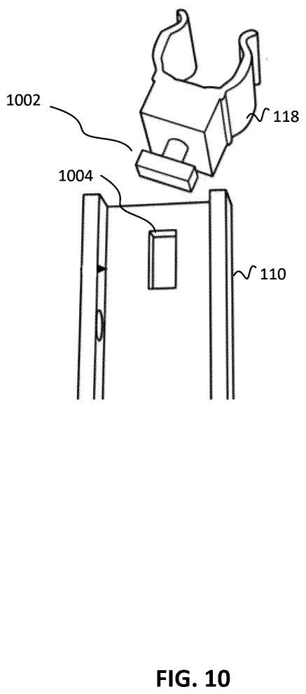

In yet some examples/embodiments, the one or more clamps 118 can have various geometries, such as a C-shaped clamp such as the ones depicted in FIGS. 1A-1B. For example, the diameter of the clamps 118 can vary depending on dimensions of the tubing to be held by the tube holding device 100. Further, the one or more clamps 118 can be fixed to the adjustable plate 110 or can be removable to and from the adjustable plate 110. By way of example, the clamps 118 can be fixedly attached to the adjustable plate 110 via fasteners, adhesive, or integral molding. In other examples, the clamps 118 can be removably secured using snap-fit, dovetail, or T-slot engagement with the plate 110, allowing replacement or repositioning. In some embodiments, a base of each clamp 118 includes a protrusion that slides into a corresponding groove on the plate 110 and is retained by a detent or set screw. FIG. 10 illustrates an example of a T-slot engagement with the plate 110. As shown in FIG. 10, the clamp 118 can include a T-shaped protrusion 1002 extending from a surface of the clamp that is opposite the holding mechanism. Additionally, the plate 110 can include a slot 1004 having a complementary shape to the T-shaped protrusion 1002. To attach the clamp 118, the clamp 118 can be oriented such that the T-shaped protrusion 1002 is aligned with the slot 1004 in the same longitudinal orientation. The T-shaped protrusion 1002 can then be inserted into the slot 1004. Then the clamp 118 can be rotated (e.g., 90 degrees), such that the T-shaped protrusion 1002 is in a latitudinal orientation and cannot pass back through the slot 1004.

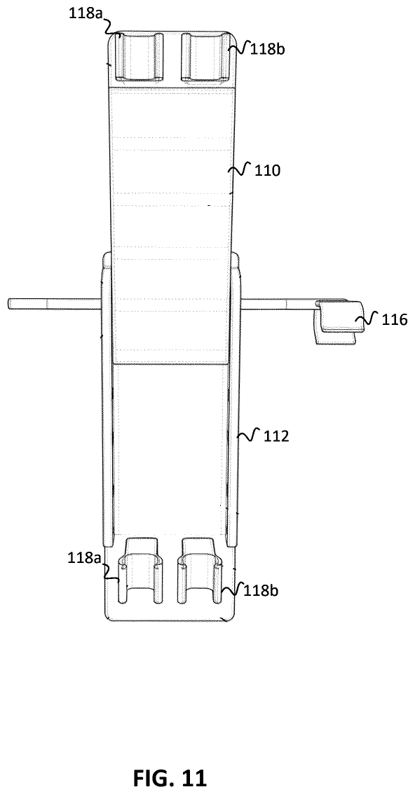

Additionally, the number of the clamps 118 attached to the adjustable plate 110 can vary. While two clamps 118 are shown in FIGS. 1A-1B, in other examples, more or less clamps can be used. Further, where a plurality of the clamps 118 are attached to the adjustable plate 110, respective clamps 118 can have different shapes and/or sizes that other clamps 118 attached to the same adjustable plate 110. In yet some examples, the one or more clamps 118 can be sized to receive medical hoses and can include semi-rigid arms configured to apply compressive force thereto when the hoses are secured, thereby preventing sagging or tangling during use. For example, FIG. 11 illustrates an example embodiment comprising two sets of clamps 118 (e.g., a first set of clamps 118a, and second set of clamps 118b).

In an example operation, the tube holding device 100 can be employed as a bedside device. For example, the tube holding device 100 can be configured in a collapsed and retracted state during storage and/or transportation to a bed, where a patient resides. Subsequently, the tube holding device 100 can be transitioned to the open state. The tapered end 108 of the base arm 102 (or at least a portion of the base arm 102 that includes the tapered end 108) can be inserted under the mattress of the bed (e.g., between the mattress and the bed platform, such as a boxspring). Thereby, at least a part of the mattress can be positioned on the front face 120 of the base arm 102. The base arm 102 can be further pushed under the mattress until the front face 120 of the support arm 104 is adjacent to a side of the mattress. While the front face 120 of the base arm 102 and support arm 104 are depicted as smooth surfaces in FIGS. 1A-1B, embodiments in which the front faces 120 are textured (e.g., with one or more friction enhancing patterns, engravings, and/or the like) are also envisaged.

As such, the weight and structure of the mattress can secure and stabilize the positioning of the tube holding device 100, whereupon the one or more clamps 118 can be adjacent to the side of the bed and ready to receive tubing that may extend from one or more medical devices in the vicinity to the patient in the bed. Additionally, the locking mechanism 116 can be disengaged, and the adjustable plate 110 can be extended to a desired height for the given tube management circumstance. Once the desired height is achieved, the locking mechanism 116 can be re-engaged to secure the new positioning of the adjustable plate 110.

In some examples, tubing from medical equipment in the vicinity can then be positioned within the one or more clamps 118 of the tubing holding device 100, whereupon the tube holding device 100 can secure the location of the tubing, inhibit tangling of the tubing, and/or reduce a chance of tripping on the tubing. While operation of the tube holding device is described in connection with a bed above, one of ordinary skill in the art will recognize that the tube holding device 100 can be employed in other context where an object can be utilized to stabilize the base arm 102.

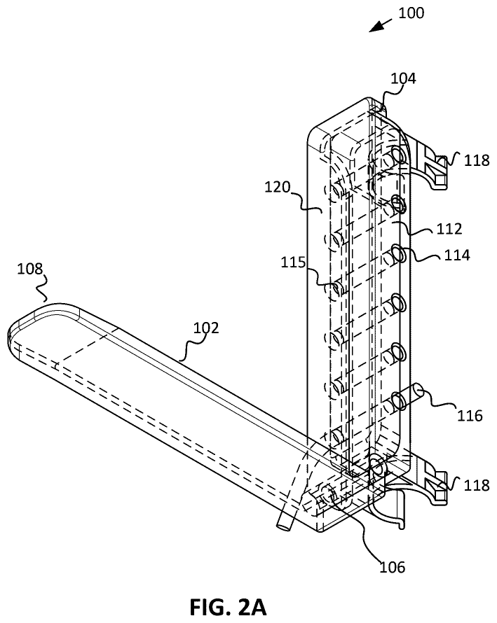

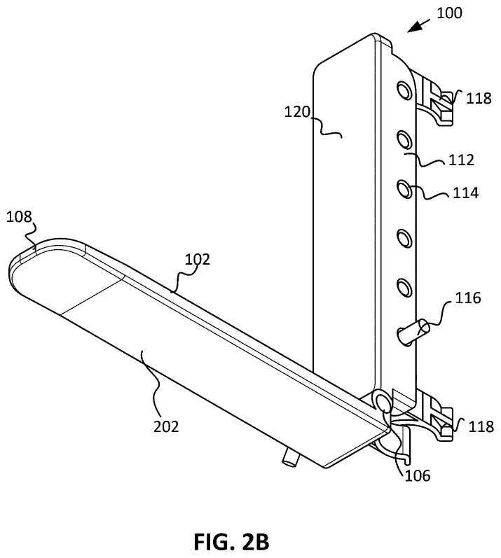

FIGS. 2A-2B depict example bottom-up perspectives of the tube holding device 100 in accordance with one or more embodiments described herein. FIG. 2A depicts the tube holding device 100 with internal components delineated by dashed lines. FIG. 2B depicts the tube holding device 100 of FIG. 2A with solid surfaces, such that internal components are not visible. As shown in FIGS. 2A-2B, a back side 202 of the base arm 102 can be substantially planar.

FIG. 3 depicts an example side view of the tube holding device 100 in accordance with one or more embodiments described herein. As shown in FIG. 3, the adjustable plate 110 can have a plurality of holes 115 that can align with each of the sets of holes 114 in the longitudinal sidewalls 112. In one or more embodiments, the adjustable plate 110 can also have a single hole 115, which can be aligned with various sets of the holes 114 in the longitudinal sidewalls 112 depending on the extension state of the adjustable plate 110.

FIG. 4 depicts an example top-down view of the tube holding device 100 in accordance with one or more embodiments described herein. As shown in FIG. 4, the base arm 102 can have a substantially rectangular shape with rounded corners to facilitate insertion under, for example, a mattress. Additionally, the longitudinal sidewalls 112 can be in close proximity to the adjustable plate 110 to reduce lateral movement of the adjustable plate 110 within the channel 150 of the support arm 104.

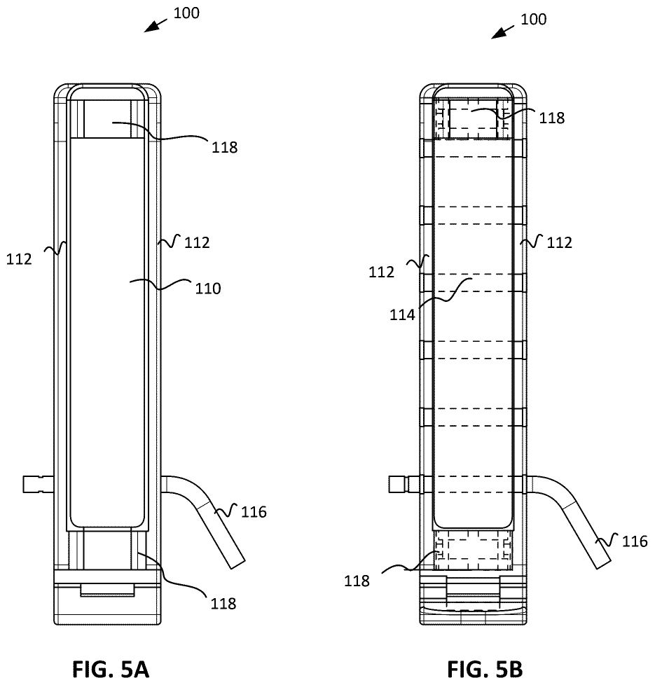

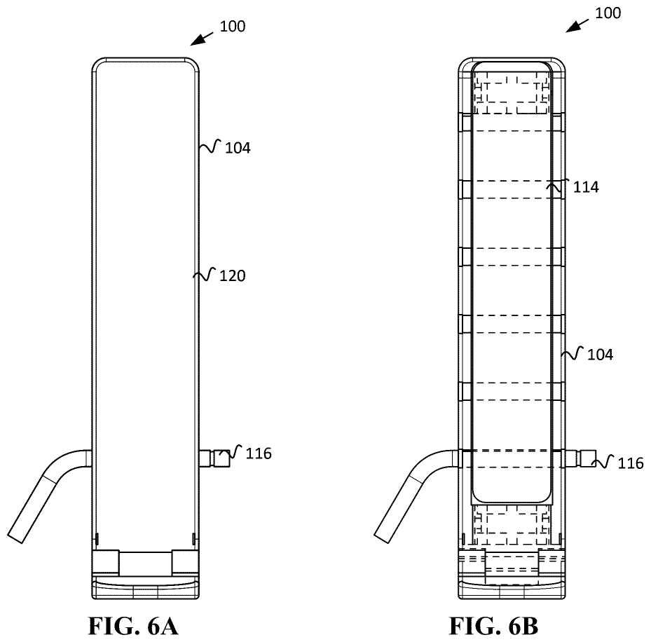

FIGS. 5A-5B depict example front views of the tube holding device 100 in accordance with one or more embodiments described herein. FIG. 5A depicts the tube holding device 100 with solid surfaces, such that internal components are not visible. FIG. 5B depicts the tube holding device 100 of FIG. 5A with internal components delineated by dashed lines. FIGS. 6A-6B depict example back views of the tube holding device 100 in accordance with one or more embodiments described herein. FIG. 6A depicts the tube holding device 100 with solid surfaces, such that internal components are not visible. FIG. 6B depicts the tube holding device 100 of FIG. 6A with internal components delineated by dashed lines.

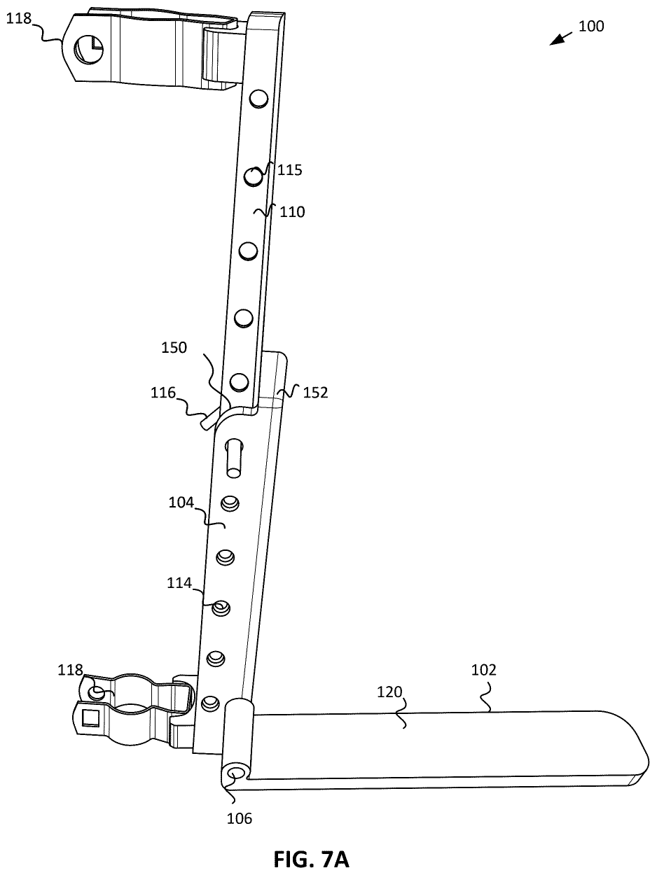

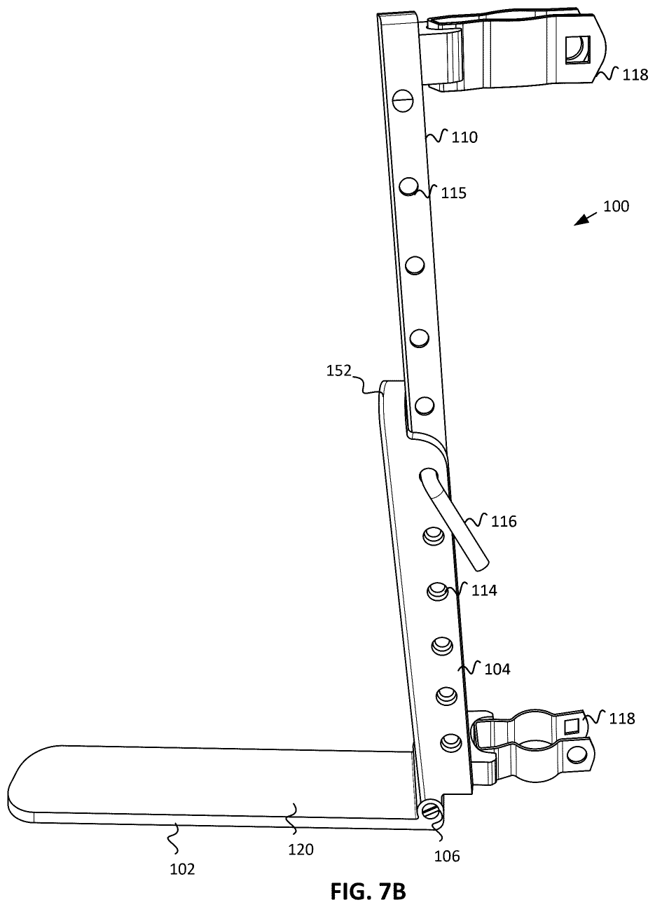

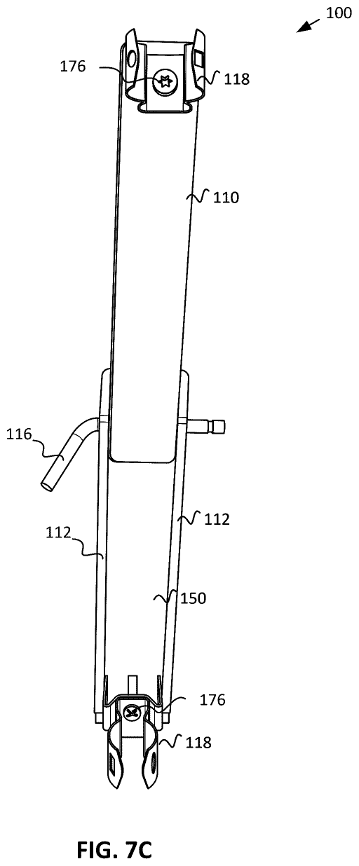

FIGS. 7A-7C illustrate example perspective views of the tube holding device 100 in an extended configuration. For example, the adjustable plate 110 can be slidably extended along a longitudinal axis of the support arm 104 such that a distal end of the adjustable plate 110 extends beyond a distal end 152 of the support arm 104 and away from the channel 150. The adjustable plate 110 can be secured at the extended position by inserting the locking mechanism 116 through aligned holes 115 in the adjustable plate 110 and a corresponding set of aligned holes 114 in the longitudinal sidewalls 112 of the support arm 104, thereby preventing further sliding or movement of the adjustable plate 110 relative to the support arm 104, achieving the extended configuration as shown in FIGS. 7A-7B. FIG. 7B illustrates the same extended configuration shown in FIG. 7A but with the tube holding device 100 oriented in an opposite direction, where the base arm 102 extends from the hinge 106 from right to left in FIG. 7B and from left to right in FIG. 7A, thereby demonstrating operability of the tube holding device 100 in either orientation.

FIG. 7C illustrates a back-side view of the tube holding device 100 in the extended configuration shown in FIGS. 7A and 7B. As shown in FIG. 7C, in some examples, the one or more clamps 118 can be secured to a front surface of the adjustable plate 110 via one or more screws 176 (e.g., threaded fasteners such as machine screws or thumbscrews) that engage corresponding threaded apertures or nuts on the back of the clamps 118 and the adjustable plate 110 and/or support arm 104, providing a robust yet removable attachment that allows for easy interchangeability. While the clamps 118 are depicted as C-shaped in various examples described herein, such as FIGS. 7A-7C, different other types of clamps or tube holders can be employed, such as spring-loaded clips, adjustable double tube clamps with extendable jaws, magnetic retainers, or hook-and-loop straps. This modular design of the tube holding device 100 thus can accommodate different tube supporting structures and support tubes of varying diameters (e.g., from ¼ inch to 2 inches or more), enabling versatile adaptation to diverse medical applications without necessitating multiple dedicated devices.

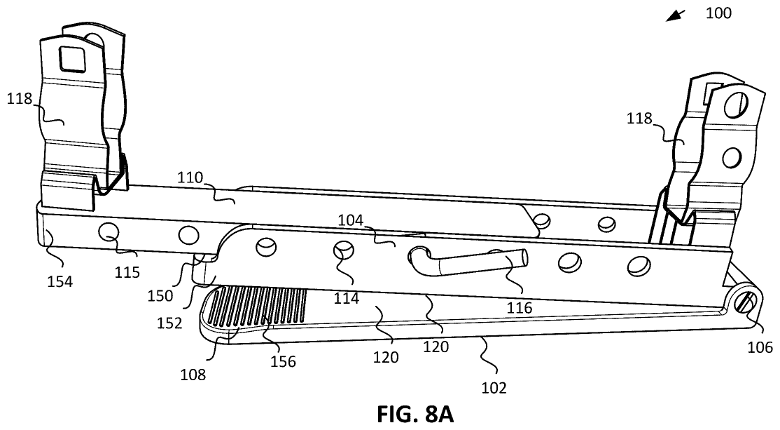

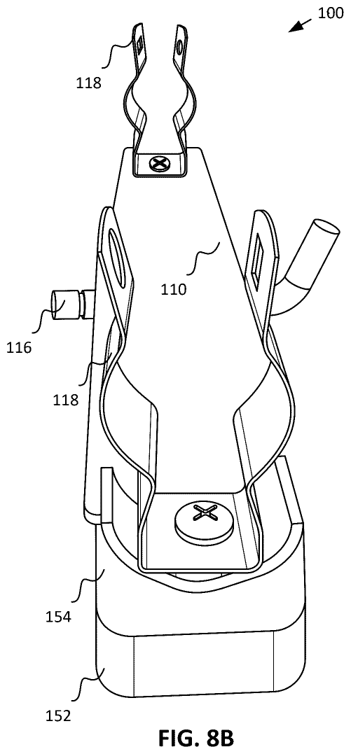

FIGS. 8A-8B depict example perspective views of the tube holding device 100 in a folded configuration. For example, the support arm 104 can be rotated about the hinge 106 relative to the base arm 102 such that the front faces 120 of the base arm 102 and support arm 104 are substantially parallel and/or adjacent. This results in a compact form factor suitable for storage and/or transportation. To achieve this folded configuration, the locking mechanism 116 can be disengaged to allow free sliding movement of the adjustable plate 110 along the channel 150. The adjustable plate 110 can be retracted into the channel 150 along the longitudinal axis of the support arm 104. Once retracted, the locking mechanism 116 can be inserted through aligned holes 115 in the adjustable plate 110 and a corresponding lower set of aligned holes 114 in the longitudinal sidewalls 112. This secures the adjustable plate 110 in the retracted position and prevents sliding or movement relative to the support arm 104. As shown in FIG. 8A, the respective distal end 154 of the adjustable plate 110 can be positioned about or near the distal end 152 of the support arm 104, with the adjustable plate 110 extending beyond the distal end 152. As shown in FIG. 8B, the distal end 154 of the adjustable plate 110 is aligned with the distal end 152 of the support arm 104, such that the adjustable plate 110 is fully engaged within the channel 150 with no overhang. As shown in FIGS. 8A-8B, in some examples, the tapered end 108 of the base arm 102 can include ridges 156, which can provide friction for gripping the support surface (e.g., a mattress) during insertion and/or deployment.

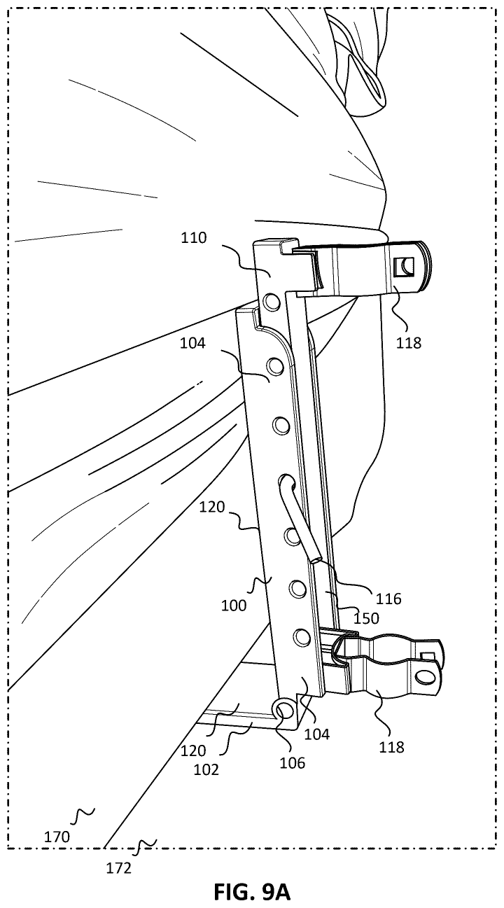

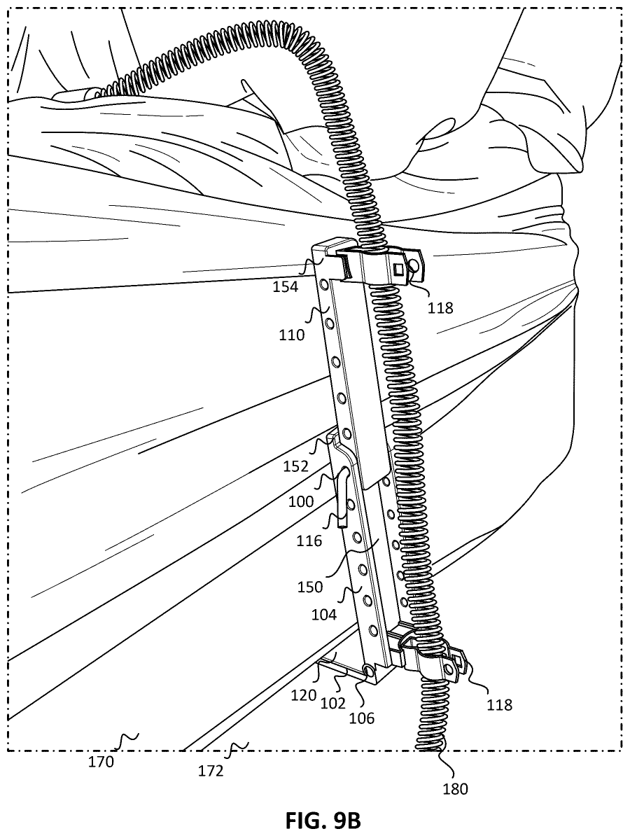

FIGS. 9A-9B illustrate example operational views of the tube holding device 100 in a deployed configuration, demonstrating integration with a support surface such as a bed for managing medical tubing. FIG. 9A depicts the tube holding device 100 in at least a partially deployed state (or deployed configuration), where the base arm 102 is inserted between a mattress 170 and a floor surface 172 to secure the tube holding device 100 in place. In this example, the base arm 102 is positioned between the mattress 170 and the floor surface 172; however, in other embodiments, the base arm 102 can be inserted between the mattress 170 and a box spring (or other bed platform) or even between two stacked mattresses. The support arm 104 abuts a side of the mattress 170, with the front face 120 of the support arm 104 thereof adjacent to the mattress edge for stability. In the example of FIG. 9A, the adjustable plate 110 can remain in a retracted position within the channel 150, secured by the locking mechanism 116 engaging a lower set of aligned holes 114 and 115, positioning the clamps 118 at a baseline height near the hinge 106. In some instances, this initial deployment allows for quick setup without immediate adjustment, leveraging a weight of the mattress 170 to anchor the base arm 102 and prevent slippage during use.

FIG. 9B depicts the tube holding device 100 in an extended configuration. To achieve the extended configuration, the locking mechanism 116 can be disengaged to slide the adjustable plate 110 upward along the longitudinal axis of the support arm 104, thereby extending the distal end 154 of the adjustable plate 110 away from the distal end 152 of the support arm 104. The locking mechanism 116 can be engaged (or re-engaged) through a selected upper set of aligned holes 114 and 115 to fix the adjustable plate 110 at a desired height. A medical tube 190, by way of example, a flexible hose from a CPAP or BiPAP machine, can be routed through the one or more clamps 118 attached to the adjustable plate 110. The tube 190 can extend upward along the side of the mattress 170 to a top portion thereof, where the tube 190 can be connected to a patient (not shown) via a mask or interface, in some instances. Because the adjustable plate 110 can be extended with respect to the support arm 104 customizable height adjustment can achieved to match varying bed dimensions, patient positions, and/or therapy needs, thereby optimizing tube routing and/or minimizing excess slack that could lead to tangling or pulling. This adjustability can improve patient comfort by reducing a risk of tube displacement during movement, preventing air leaks or therapy interruptions, and eliminating trailing hazards across the bed surface or floor that could cause tripping for caregivers or the patient. Furthermore, in some instances, use of semi-rigid C-shaped clamps as the one or more clamps 118 can secure hold the tube 190 in place by applying sufficient compressive force upon insertion, flexing to accommodate a tube's diameter while maintaining grip to inhibit sagging, kinking, and/or unintended disconnection, without restricting airflow or causing undue stress on the tubing. Thus, the tube holding device 100 can allow for reliable, non-intrusive tube management, promoting safer and more effective in-home or bedside medical treatments.

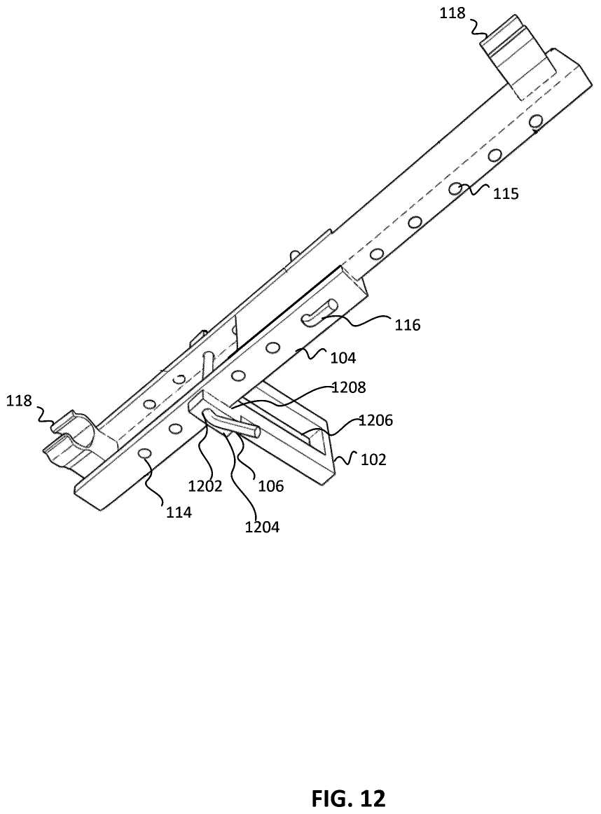

FIG. 12 illustrates a further example embodiment of the tube holding device 100 in accordance with one or more embodiments described herein. As shown in FIG. 12, the pin 106 can be a removable pin similar to the locking pin 116. For example, the pin 106 can establish the rotatable connection between the base arm 102 and the support arm 104 by being inserted into one or more holes (114, 1202) in the support arm 104 and the base arm 102. For instance, the base arm 102 can include one or more integrated sections 1204 that extend adjacent to the sidewalls of the support arm 104. Each of the integrated sections 1204 can include at least one hole 1202. The holes 1202 of the base arm 102 can be aligned with one or more sets of holes 114 in the support arm 104. Once aligned, the pin 106 can be inserted through the holes 114, 1202 to join the base arm 102 and support arm 104. Advantageously, this embodiments enables the pivot point between the base arm 102 and the support arm 104 to be adjustable to various elevations depending on which set of holes 114 is engaged. Further, as shown in FIG. 12, the base arm 102 can have a numerous shapes and/or geometries. FIG. 12 shows an example geometry comprising a relief slot extending the central, longitudinal axis of the base arm 102. The relief slot 1206 can enable the integrated sections 1204 to flex so as to accommodate the width of the support arm 104. Once coupled, the support arm 104 can be sandwiched between the integrated sections 1204 and positioned on a ledge 1208 located where the integrated sections 1204 extend from the body of the base arm 102. For example, the ledge 1208 can inhibit rotation of the support arm 104 in relation to the base arm 102, thereby providing additional stability to the tube holding device 100 in the open configuration.

Several aspects of the present technology are set forth in the following numbered examples.

Example 1. A tube holding apparatus comprising: a base arm rotatably coupled to a support arm, the support arm having a front side that faces toward the base arm and a back side that faces opposite the front side; a channel positioned on the back side of the support arm, the channel housing an adjustable plate; and one or more clamps attached to the adjustable plate, wherein the one or more clamps are configured to hold one or more tubes.

Example 2. In some implementations of Example 1, the base arm is coupled to the support arm via a hinge that enables rotation of the support arm relative to the base arm to transition the apparatus between a folded configuration and a deployed configuration.

Example 3. In some implementations of Example 2, in the folded configuration, front faces of the base arm and the support arm are substantially parallel and adjacent, and in the deployed configuration, the base arm extends substantially perpendicular to the support arm.

Example 4. In some implementations of any of Examples 1-3, the channel is at least partially defined by longitudinal sidewalls, and the adjustable plate is slidable along the channel in a longitudinal direction of the support arm.

Example 5. In some implementations of Example 4, the longitudinal sidewalls include a plurality of aligned sets of holes spaced along the longitudinal direction.

Example 6. In some implementations of Example 5, the adjustable plate includes at least one transverse through-hole configured to align with a selected set of the aligned sets of holes when the adjustable plate is positioned at a desired height along the channel.

Example 7. In some implementations of Example 6, further comprising a locking mechanism insertable through the selected set of the aligned sets of holes and the at least one transverse through-hole to secure the adjustable plate at the desired height.

Example 8. In some implementations of Example 7, the locking mechanism is a pin that extends through the longitudinal sidewalls and the adjustable plate.

Example 9. In some implementations of any of Examples 1-8, the one or more clamps comprise a plurality of clamps, each clamp having a C-shape configured to apply compressive force to a tube inserted therein.

Example 10. In some implementations of Example 9, at least two of the plurality of clamps have different inner diameters configured to accommodate tubes of varying sizes.

Example 11. In some implementations of any of Examples 1-10, the one or more clamps are removably attached to the adjustable plate via at least one of snap-fit, dovetail, or T-slot engagement.

Example 12. In some implementations of any of Examples 1-11, the base arm includes a tapered distal end configured to facilitate insertion beneath a support surface.

Example 13. In some implementations of any of Examples 1-12, the front side of the support arm and a front face of the base arm include a friction-enhancing texture.

Example 14. In some implementations of any of Examples 1-13, the hinge includes a locking mechanism configured to selectively lock an angle between the base arm and the support arm.

Example 15. A foldable bedside tube management device comprising: a base arm rotatably coupled to a support arm via a hinge; a channel positioned on a back side of the support arm and defined by longitudinal sidewalls including a plurality of aligned sets of holes; an adjustable plate housed within the channel and having at least one transverse through-hole; one or more C-shaped clamps coupled to the adjustable plate and extending away from a front side of the support arm; and a locking pin insertable through a selected set of the aligned sets of holes and the at least one transverse through-hole to fix a height of the adjustable plate along the channel; wherein the hinge enables transition between a folded configuration in which the base arm and support arm are substantially parallel and a deployed configuration in which the base arm extends substantially horizontally for insertion beneath a mattress and the support arm extends substantially vertically.

Example 16. In some implementations of Example 15, the adjustable plate is slidable from a retracted position in which a distal end of the adjustable plate is substantially aligned with a distal end of the support arm to an extended position in which the distal end of the adjustable plate extends beyond the distal end of the support arm.

Example 17. In some implementations of Example 15 or 16, the one or more C-shaped clamps comprise a plurality of clamps, at least one of which is removably attached to the adjustable plate.

Example 18. In some implementations of any of Examples 15-17, the base arm includes a tapered distal end and a substantially planar front face configured to be positioned beneath the mattress.

Example 19. In some implementations of any of Examples 15-18, the one or more C-shaped clamps are semi-rigid and configured to hold medical hoses for continuous positive airway pressure or bilevel positive airway pressure therapy.

Example 20. A tube holding apparatus comprising: a base arm having a tapered distal end and rotatably coupled via a hinge to a support arm, the support arm having a front side facing the base arm and a back side opposite the front side; a channel on the back side defined by longitudinal sidewalls with multiple aligned hole sets; an adjustable plate slidably housed in the channel, the adjustable plate including a transverse through-hole alignable with any of the multiple aligned hole sets; a plurality of clamps attached to the adjustable plate, each clamp configured to secure a medical tube; and a locking mechanism configured to engage a selected aligned hole set and the transverse through-hole to lock the adjustable plate at a user-selected height.

Terms of orientation are used herein merely for purposes of convention and referencing and are not to be construed as limiting. However, it is recognized that these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, as used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the technology. In addition, many modifications will be appreciated by those skilled in the art to adapt a particular instrument, situation, or material to embodiments of the disclosure without departing from the essential scope thereof. Therefore, it is intended that the technology is not limited to the particular embodiments disclosed, or to the best mode contemplated for carrying out this technology, but that the technology will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What is claimed is:

1. A tube holding apparatus, comprising:
a base arm rotatably coupled to a support arm, the support arm having a front side that faces toward the base arm and a back side that faces opposite the front side;
a channel positioned on the back side of the support arm, the channel housing an adjustable plate; and
one or more clamps attached to the adjustable plate, wherein the one or more clamps are configured to hold one or more tubes.

2. The tube holding apparatus of claim 1, wherein the base arm is coupled to the support arm via a hinge.

3. The tube holding apparatus of claim 2, comprising a plurality of the clamps.

4. The tube holding apparatus of claim 3, wherein the clamps are C-clamps.

5. The tube holding apparatus of claim 1, wherein the adjustable plate is configured to traverse the channel along a longitudinal axis of the support arm.

6. The tube holding apparatus of claim 1, wherein the channel is at least partially defined by sidewalls that have one or more aligned sets of holes.

7. The tube holding apparatus of claim 6, wherein the adjustable plate has at least one hole that travels the entirety of the adjustable plate along a transverse direction.

8. The tube holding apparatus of claim 7, further comprising a locking mechanism that interfaces with at least one set of the set of holes of the sidewalls and the at least one hole of the adjustable plate to secure a position of the adjustable plate within the channel.

9. The tube holding apparatus of claim 8, wherein the locking mechanism is a pin that extends through the sidewalls and through the adjustable plate.

10. A method of managing medical tubing using a tube holding device having a base arm rotatably coupled to a support arm, a channel on a back side of the support arm housing an adjustable plate, and one or more clamps on the adjustable plate, the method comprising:
transitioning the tube holding device from a folded configuration to a deployed configuration by rotating the support arm relative to the base arm;
inserting the base arm beneath a support surface such that the support arm abuts a side thereof; and
securing one or more medical tubes within the one or more clamps.

11. The method of claim 10, further comprising:
adjusting a height of the adjustable plate within the channel along a longitudinal axis of the support arm; and
inserting a locking pin through aligned holes in sidewalls of the channel and the adjustable plate to fix the plate at the adjusted height.

12. The method of claim 11, wherein the adjustable plate is adjusted from a retracted position to an extended position such that a distal end of the plate extends beyond a top of the support arm.

13. The method of claim 10, wherein the one or more medical tubes are continuous positive airway pressure or bilevel positive airway pressure hoses.

14. The method of claim 10, wherein the base arm includes a tapered distal end to facilitate insertion beneath the support surface.

15. A foldable bedside holder, comprising:
a base arm rotatably coupled to a support arm via a hinge;
a channel positioned on a back side of the support arm and defined by sidewalls including one or more aligned sets of holes, the channel housing an adjustable plate with at least one through-hole;
one or more clamps coupled to the adjustable plate; and
wherein the hinge enables rotation of the support arm relative to the base arm to transition the holder between a folded configuration and a deployed configuration, and in the deployed configuration, the adjustable plate is slidable along the channel to a selected height and securable via a locking mechanism insertable through a selected set of the aligned holes and the at least one through-hole.

16. The foldable bedside holder of claim 15, wherein the one or more clamps are C-shaped and extend from the adjustable plate in a direction away from a front side of the support arm.

17. The foldable bedside holder of claim 15, wherein the one or more clamps are removably attached to the adjustable plate via at least one of snap-fit, dovetail, or T-slot engagement.

18. The foldable bedside holder of claim 15, wherein in the folded configuration, front faces of the base arm and the support arm are substantially parallel and adjacent, and in the deployed configuration, the base arm extends horizontally for insertion beneath a support surface while the support arm extends vertically to position the adjustable plate and clamps at a selectable height adjacent to the support surface.

19. The foldable bedside holder of claim 15, wherein the base arm includes a tapered distal end configured to facilitate tool-free insertion beneath the support surface.

20. The foldable bedside holder of claim 15, wherein the one or more clamps are sized to receive medical hoses and include semi-rigid arms configured to apply compressive force thereto when the hoses are secured.
